(19) United States
(12) Patent Application Publication
(10) Pub. No.: US 2010/0105981 A1
(43) Pub. Date: Apr. 29, 2010

(54) Puncturing Instrument

(75) Inventors: Chi-Nghia HO, Stuttgart (DE); Marc O. Schurr, Tubingen (DE)

Correspondence Address: Sunstein Kann Murphy & Timbers LLP, 125 SUMMER STREET, BOSTON, MA, 02110-1618, US

(73) Assignee: Ovesco Endoscopy AG, Tubingen (DE)

(21) Appl. No.: 12/603,178

(22) Filed: Oct. 21, 2009

(30) Foreign Application Priority Data
Oct. 27, 2008 (DE) 102008043207.5

(51) Int. Cl. A61B 17/34; A61B 1/018

(52) U.S. Cl. 600/104; 606/185

(57) ABSTRACT

The invention relates to a puncturing instrument comprising a guiding tube at the distal end of which an expanding anchor means is disposed and a protective sleeve enclosing at least the distal end portion of the guiding tube especially in the area of the expanding anchor means to be axially displaceable, wherein a hollow needle or a gastroscope having a distal end in the form of a cutting edge is supported to be axially movable inside or along the guiding tube.

The present invention relates to a puncturing instrument preferably for gastroscopic insertion to provide a through passage preferably through the gastric wall.

From prior art in accordance with US 2007/0043380 A1, for instance, a puncturing instrument of this generic type is known. Inter alia, this instrument comprises a flexible guiding tube in which a gastroscope is supported to be axially movable. The gastroscope has at its distal end a cutting edge by which the tissue material can be severed. Moreover, in the guiding tube syringes in which respective anchoring wires are slide-mounted are formed in an insulating manner. They can exit at the distal end portion of the guiding tubes to twist anchor-like. For this purpose, the anchoring wires are made of a material having a memory effect, as it is called, or a spring-elastic material adopting a hook shape in the relieved stated.

In order to provide a through hole through the gastric wall by said known puncturing instrument, the guiding tube is inserted gastroscopically into the stomach and is positioned in a predefined area at the inside of the gastric wall. Then the anchoring wires are axially displaced in the direction of the distal end of the guiding tube and exit the distal end of the guiding tube. In so doing, the anchoring wires penetrate the gastric wall and interlock there. In this way, the guiding tube is fixed to the inner gastric wall. After that, the gastroscope is forced through the guiding tube to the front, the distal cutting edge of the gastroscope cutting the gastric wall. Finally the guiding tube can be pushed through the orifice produced by the gastroscope so as to pack the perforation of the stomach.

The afore-described design consequently requires providing syringes separated from each other to accommodate the anchoring wires, which in total renders the manufacture of the guiding tubes more complicated and more expensive. In addition, the anchoring wires have to be guided in the syringes frictionless, if possible, which makes high demands to the surface finish of both the wires and the syringes.

In view of these facts, it is the object of the present invention to provide a generic puncturing instrument that is adapted to be manufactured in a simpler and more inexpensive manner and, moreover, ensures a more reliable function.

This object is achieved by a puncturing instrument comprising the features of claim 1. Further advantageous configurations of the invention are the subject matter of the other subclaims.

Consequently, the core of the invention consists in providing the guiding tube itself with an anchor means, preferably by an integral design or tightly fixed at the distal end thereof and in covering said anchor means with a protective shell. If, accordingly, the protective shell is removed, for instance by retracting the protective shell or by advancing the guiding tube, the anchor means unfolds preferably automatically and is anchored in the tissue of the patient.

This design principle can be put into practice by far more roughly than in the prior art, that is to say the individual functional members exhibit a less fragile structure and therefore can be manufactured in a cheaper way. Moreover, vis-à-vis the cited prior art larger tolerances are permitted concerning the dimensional stability of the individual members so that the operability is increased on the whole.

Hereinafter the invention is illustrated in detail by way of a preferred embodiment with reference to the accompanying drawings.

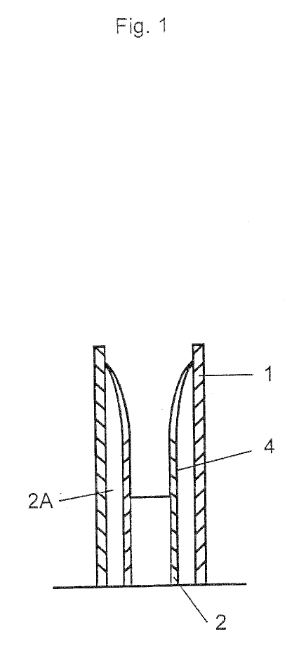

FIG. 1 shows the longitudinal section of a distal end portion of the puncturing instrument according to the invention in the retracted position, i.e. in a state suited for gastroscopic insertion into the interior of a stomach,

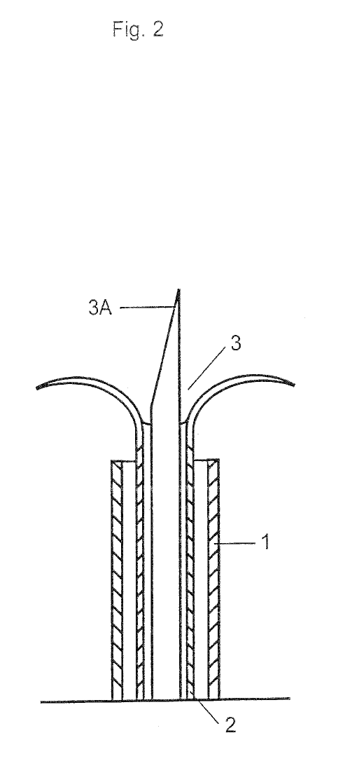

FIG. 2 shows the puncturing instrument according to the invention in the extended state, i.e. a state in which a gastric wall passage can be provided and

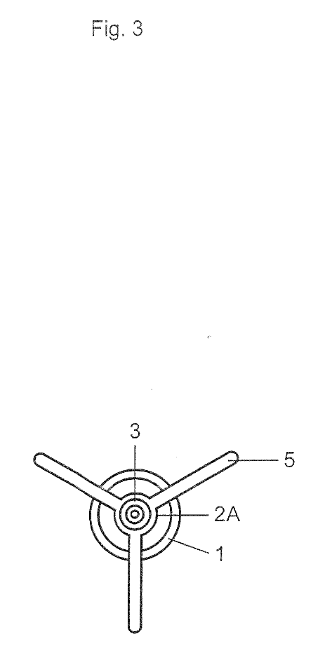

FIG. 3 shows a top view of the puncturing instrument according to the invention in the extended state.

In accordance with FIG. 1, the puncturing instrument according to the invention comprises, as shown in a preferred embodiment of the invention, a guiding tube 2 at the distal end of which a kind of expanding anchor 4 is preferably integrally formed and an anchor or protective sleeve 1 which encloses at least the distal end portion of the guiding tube 2 especially in the area of the expanding anchor 4 in a manner to be axially displaceable. (By saying that the sleeve “encloses” the tube, it is meant that the sleeve surrounds or encompasses the tube such that the sleeve may be shiftably mounted on the outer side of the tube.) Inside the guiding tube 2 a hollow needle or a gastroscope including a distal end in the form of a cutting edge is supported in an axially or axially parallel movable way. As an alternative to this, it is also possible, in case that the expanding anchor has especially small dimensions, to guide the hollow needle or the gastroscope outside the guiding tube in parallel thereto, wherein the hollow needle or the gastroscope in this case is preferably directly enclosed by the protective sleeve.

In detail, the expanding anchor 4 consists of preferably three needles or claws 5 arranged at equal circumferential distances which in the relieved state are bent radially outwardly in a spring-elastic fashion. The needles or tabs 5 are integrally converted at the roots thereof into a sleeve-shaped base member 2A of the expanding anchor 2, thereby providing a sleeve-shaped cavity. The sleeve member 2A further is connected preferably integrally to the distal end of the guiding tube 2 in a coaxial, preferably joint-to-joint manner so as to form a continuous guiding passage.

Inside the guiding passage a hollow needle or a gastroscope 3 extending along the guiding tube 2 is provided axially or axially parallel (i.e. off-center) movable relative to the guiding tube 2. The hollow needle or the gastroscope 3 as well as the guiding tube 2 are shown to be flexible so that they can be advanced, for instance, through the esophagus of a patient into the stomach. The hollow needle or the gastroscope 3 includes at its distal end a cutting edge 3A for severing tissue material, e.g. of a gastric wall.

The sleeve 1 enclosing at least the expanding anchor 4 is likewise supported to be axially movable with respect to the guiding tube 2 and extends along the guiding tube 2 as well as the hollow needle or the gastroscope 3 located therein. Preferably the outer sleeve 1 extends over the entire length of the guiding tube 2 so that it is operable at the proximal end of the puncturing instrument. However, as an alternative it is also possible that the guiding sleeve 1 extends axially along the expanding anchor 4 merely in the distal end portion and is operable by a push-pull element extending axially parallel to the guiding tube 2, for instance a Bowden cable.

The function of the puncturing instrument according to the invention can be paraphrased as follows.

The puncturing instrument can be inserted through the esophagus into the stomach of a patient by virtue of the flexibility of the guiding tube 2, the hollow needle or the gastroscope 3 supported therein and the sleeve 1 enclosing the guiding tube. As soon as the distal end of the puncturing instrument has reached the gastric wall at a predetermined position and contacts the same, respectively, the guiding tube 2 is further advanced, wherein the sleeve 1 enclosing the guiding tube 2 at least at the distal end portion thereof is withheld, however. Accordingly, the needles or tapered tabs 5 of the expanding anchor 4 are axially exposed at the distal end of the guiding tube 2, i.e. they protrude continuously from the distal end of the protective sleeve 1, wherein upon further advancing of the guiding tube 2 the needles or tabs 5 spread radially outwardly due to the spring bias or a “memory effect” in the material. During this movement the needles or tabs 5 penetrate the tissue of the gastric wall and anchor there due to their continuously radially spreading deformation. Consequently, in the completely extended state the needles or tabs 5 have a circular arc shape, whereby tensile or compression force can be introduced to the gastric wall in axial direction of the guiding tube 2. That is to say that, for example, the gastric wall can be pulled inwardly through the guiding tube 2, whereby at the outside of the gastric wall a cavity is formed in the spreading area of the needles or tabs 5 of the expanding anchor.

Ultimately, the hollow-shaped needle or the gastroscope 3 is advanced inside the guiding tube 2, the cutting edge cutting into the gastric wall tissue at the distal end of the hollow needle or the gastroscope so as to provide a through orifice from the inside of the gastric wall to the outside of the gastric wall. By reason of the special shape of the expanding anchor, especially of the spring-elastic needles or tabs which radially spread automatically upon advancing the expanding anchor, it is possible, as already described in the foregoing, to withdraw the gastric wall so as to form a circular cavity at the outside of the gastric wall. In this way the surrounding tissue can be prevented from being injured upon piercing the gastric wall by means of the hollow needle or the gastroscope including the cutting edge.

The above description illustrates that equipping the guiding tubes with the expanding anchor makes the formation of additional guiding syringes inside the guiding tube redundant. In this manner, the guiding sleeve can be reduced to a simple geometry and thus a simple way of manufacture. Moreover, by arranging a sleeve or cap enclosing at least the expanding anchor it is ensured that in the retracted state of the expanding anchor an injury of tissue is safely avoided. In addition, the manufacture of such sleeve or protective cap/sleeve is simple and thus inexpensive. It has finally turned out that by virtue of the less filigree mode of construction of all components compared to prior art the reliability can be increased and thus the functionality of the instrument can be improved.

In this context, also some modifications of the afore-described first embodiment shall be described:

The expanding anchor is formed integrally with the guiding sleeve in the present embodiment. As an alternative to this it is also possible, however, to provide the expanding anchor as a component part manufactured separately from the guiding tube which is subsequently screwed, welded, soldered or glued to the distal end of the guiding tube. The fixing can be effectuated joint-to-joint or in an overlapping manner.

The needles or tips or sharp-edged tabs of the expanding anchor are spring-elastic in the preferred embodiment. As an alternative, also a material having a so-called memory effect can be used, however.

1. A puncturing instrument comprising a guiding tube at the distal end of which an expanding anchor means is disposed and a protective sleeve which encloses at least the distal end portion of the guiding tube in a manner to be axially displaceable, wherein a hollow needle or a gastroscope having a distal end in the form of a cutting edge is supported to be axially movable inside or along the guiding tube.

2. A puncturing instrument comprising a guiding tube at the distal end of which an expanding anchor means is disposed and a protective sleeve which encloses at least the distal end portion of the guiding tube in the area of the expanding anchor means in a manner to be axially displaceable, wherein a hollow needle or a gastroscope having a distal end in the form of a cutting edge is supported to be axially movable inside or along the guiding tube.

3. A puncturing instrument according to claim 2, characterized in that the expanding anchor means consists of a cup- or sleeve-shaped base member at which axially extending needles or acute tabs are formed which are preformed into a radially outwardly curved shape either spring-elastically or according to the principle of a memory effect.

4. A puncturing instrument according to claim 3, characterized in that the guiding tube is made of a flexible material.

5. A puncturing instrument according to claim 3, characterized in that the expanding anchor means is formed integrally with the guiding tube.

6. A puncturing instrument according to claim 5, characterized in that the guiding tube is made of a flexible material.

7. A puncturing instrument according to any one of the preceding claims, characterized in that the hollow needle forms the distal end piece of an elastic hose member extending to be axially displaceable inside the guiding tube and the expanding anchor means.

8. A puncturing instrument according to claim 7, characterized in that the protective sleeve is made of a flexible material and extends along the guiding tube.

9. A puncturing instrument according to any one of claims 2 to 6, characterized in that the protective sleeve is made of a flexible material and extends along the guiding tube.

10. A puncturing instrument according to any one of the claims 2 to 6, characterized in that at the proximal end of the protective sleeve a push-pull element is arranged.

11. A puncturing instrument according to claim 10, characterized in that the push-pull element is an actuating wire extending along the guiding tube.

12. A puncturing instrument according to claim 1, characterized in that the hollow needle or the gastroscope is supported along the guiding tube outside thereof but inside the protective sleeve.

13. A puncturing instrument according to claim 1, characterized in that the guiding tube is made of a flexible material.

14. A puncturing instrument according to claim 1 characterized in that the protective sleeve is arranged to surround or encompass exclusively the distal end of the guiding tube in the area of the expanding anchor means.
